(19) United States
(12) Patent Application Publication
(10) Pub. No.: US 2005/0119599 A1
(43) Pub. Date: Jun. 2, 2005

(54) Supplemental port for catheter perfusion of surgical site and methods of use

(75) Inventors: William Russell Kanz, Sacramento, CA (US); Walid Najib Aboul-Hosn, Fair Oaks, CA (US)

Correspondence Address: RYAN KROMHOLZ & MANION, S.C., POST OFFICE BOX 26618, MILWAUKEE, WI, 53226, US

(73) Assignee: A-Med Systems, Inc.

(21) Appl. No.: 10/991,255

(22) Filed: Nov. 17, 2004

Related U.S. Application Data
(62) Division of application No. 09/641,012, filed on Aug. 16, 2000.
Application No. 09/641,012 is a continuation-in-part of application No. 09/313,268, filed on May 18, 1999, now abandoned.

(51) Int. Cl. A61M 37/00

(52) U.S. Cl. 604/6.11; 604/6.14

(57) ABSTRACT

A blood pump having a supplemental outflow port and/or a supplemental inflow port. A supplemental outflow port can be used to supply blood to regions of the body during heart bypass operations, such as to perfuse heart tissue downstream from an anastomosis site during CABG procedures so as to reduce the damage to the heart tissue. A supplemental inflow port can be used to infuse blood and/or various other fluids or compositions into the patient's blood stream, such as may be helpful or advantageous during emergency situations in cardiac surgery.

### RELATED APPLICATIONS

This application is divisional of co-pending U.S. patent application Ser. No. 09/641,012, filed Aug. 16, 2000, which is a continuation-in-part of U.S. application Ser. No. 09/313,268, filed May 18, 1999, now abandoned.

### FIELD OF THE INVENTION

The present invention relates generally to blood pumps for use during heart surgery. More specifically, the present invention involves providing a supplemental port on a blood pump for delivering blood to a surgical site via a catheter or cannula arrangement to perfuse the tissue downstream from the surgical site.

### BACKGROUND OF THE INVENTION

During open heart surgery and in some emergency cardiopulmonary situations, it is necessary to have some means to bypass the heart with a blood pump. The bypass circuit may be used to completely replace the function of the heart or it may be employed to assist the heart. Typically in a bypass circuit, an inflow cannula is placed within the left ventricle and an outflow cannula is placed within the aorta. Bypass surgery typically is used to repair damaged or occluded vessels on the heart. To repair a vessel or occlusion, the surgeon usually will graft a new vessel that will supply blood to the affected area. Before applying the graft, the surgeon will occlude the target vessel proximally to the damaged area. One problem with doing this is that healthy tissue beyond or downstream from the damaged area no longer receives sufficient blood or oxygen during the operation.

Typically, such a circuit will be used for cardiopulmonary arterial bypass graph (CABG) surgery to support or supplement the heart. While CABG surgery may be accomplished on a beating heart or a still heart, the trend is moving towards beating heart surgery because it is less traumatic to the patient. When conducting beating heart CABG surgery, the patient's vessels and arteries require a replenished flow of oxygenated blood in order for the tissues to sustain without damage. When the surgeon is performing an anastomosis, the target vessel is occluded proximally to the surgical site. Problems associated with occluding the vessel include damage to tissue distal the anastomosis site. In extreme cases, the patient will require a second surgery to correct complications that were created by the first surgery.

The present invention is directed at overcoming, or at least reducing the effects of, one or more of the problems set forth above.

### SUMMARY OF THE INVENTION

The present invention concerns a blood pump with a supplemental outflow port(s). A catheter can be attached to the supplemental port at a proximal end, while the distal end of the catheter may be placed where it is desired to have a supplemental blood flow.

During CABG surgery, typically one or more of the patient's vessels are occluded. Once the vessel is occluded, the surgeon may make an anastomosis beyond the occlusion. Typically, the vessel that was occluded does not have any blood flowing through it. One prior art way to remedy this problem is to insert a stent in the area where the anastomosis is going to be placed. Unfortunately, the stent may occupy a large cross-sectional area of the vessel, reducing the overall flow through the vessel such that the area distal to the stent does not receive sufficient oxygenated blood.

This supplemental outflow port of the present invention eliminates the need for a stent and provides for a continuous source of oxygenated blood and therefore may reduce the post-surgical damage to the surrounding tissue after an anastomosis has been performed.

The present invention also concerns a supplemental inflow port for use with a blood pump. The supplemental input port can be used to input blood from an area other than the main inflow region. For example, blood removed from the heart can be filtered and then introduced back into the patient through the supplemental inflow port.

In one broad aspect of the present invention, an apparatus is provided comprising a blood pump, a main inflow port operably connected to the blood pump, a main outflow port operably connected to the blood pump, and a supplemental port operably connected to the blood pump.

In one embodiment, the supplemental port is a supplemental outflow port.

In one embodiment, the supplemental outflow port is connected to a catheter adapted to supply blood to perfuse a vein or artery.

In one embodiment, the supplemental outflow port is connected to a cannula adapted to be positioned in the patient's aorta.

In one embodiment, the supplemental port is a supplemental inflow port.

In one embodiment, the supplemental inflow port is connected to a catheter connected to a supply of blood.

In one embodiment, the supply of blood is connected to a catheter adapted to be positioned in the body to remove blood from the patient.

In one embodiment, the apparatus further comprises a valve at the supplemental port.

In one embodiment, the main inflow port is connected to a cannula adapted to be positioned in a patient's atrium or ventricle.

In one embodiment, the main outflow port is connected to a cannula to be positioned in a patient's aorta.

In one embodiment, the main outflow port is connected to a cannula to be positioned within a patient's artery.

In one embodiment, the blood pump is connected to an oxygenator.

In another broad aspect of the present invention, an apparatus is provided comprising a blood pump including a main inflow port, a main outflow port, and a supplemental outflow port. The apparatus also includes a perfusion catheter connected to the supplemental outflow port, the catheter adapted to supply blood to an artery on the heart during a bypass operation on that artery.

In a still further broad aspect of the present invention, a method is provided comprising the steps of: (a) operably connecting a blood pump to a patient; (b) pumping blood from one part of the heart to another part of the heart; and (c) supplying blood through a supplemental port on the blood pump to an artery one the heart during a bypass operation to that artery.

In one embodiment, the blood passes through an oxygenator.

In one embodiment, the blood is supplied from the supplemental port to the artery through a catheter.

In one embodiment, the blood is supplied from the supplemental port to the artery through a cannula.

### BRIEF DESCRIPTION OF THE DRAWINGS

FIG. 1 is a top view of a centrifugal blood pump having a supplemental outflow port in accordance with the present invention;

FIG. 2 is a side view of a heart (in partial cross-section) illustrating the use of a pump and cannula system having a perfusion catheter coupled to the supplemental outflow port in accordance with the present invention;

FIG. 3 is a side view of a vessel illustrating the positioning of the distal end of the perfusion catheter to deliver blood downstream from the surgical site according to the present invention; and;

FIG. 4 is a side view of a centrifugal blood pump and cannula system having a supplemental inflow port for receiving blood from a reservoir.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

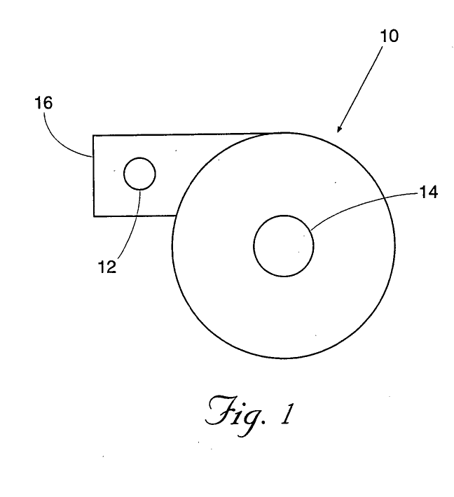

The present invention is directed at providing an improved device and related methods for delivering blood and/or other fluids to perfuse tissue and/or organs located downstream from a surgical site. Referring initially to FIG. 1, this is accomplished in one basic embodiment by equipping a pump 10 with a supplemental outflow port 12 in addition to the main fluid inflow 14 and main fluid outflow 16 traditionally found in pumps. By way of example only, the pump 10 is presented as a centrifugal blood pump well known in the art. The pump 10 normally operates under the direction of a motor (not shown) which drives an internally disposed impeller (not shown) so as to transport blood from the main fluid inflow port 14 in a generally tangential fashion out the main outflow port 16. In accordance with one embodiment of the present invention, the supplemental port 12 is formed on the structure defining the main fluid outflow port 16. As such, blood may be simultaneously directed through both the main outflow port 16 and the supplemental outflow port 12. As will be explained in greater detail below, when conducting an anastomosis or other surgical procedure that requires a supply of oxygenated blood, a cannula or catheter attached to the supplemental outflow port 12 may be employed to supply a pressurized flow of blood to (or downstream from) a surgical site.

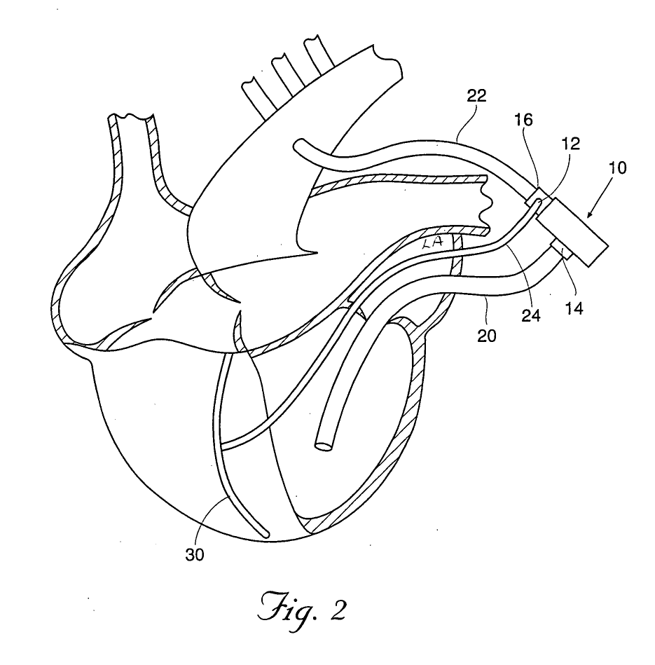

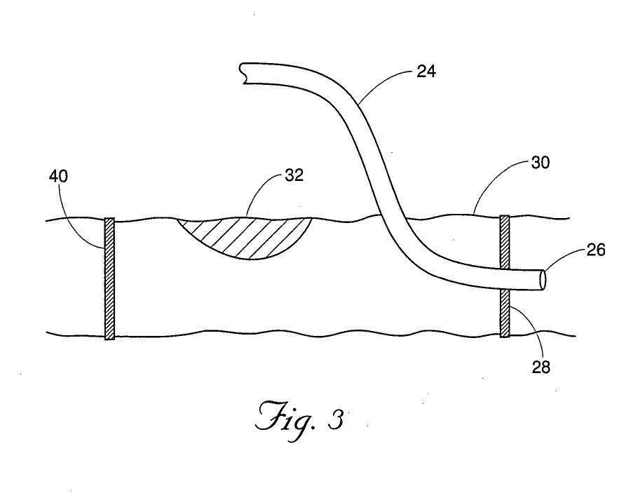

Referring to FIG. 2, the pump 10 having the supplemental outflow port 12 according to the present invention is illustrated in use as part of a pump and cannula arrangement for providing left-heart assist. More specifically, an inflow cannula 20 is coupled to the main inflow port 14, an outflow cannula 22 is coupled to the main outflow port 16, and a perfusion catheter or cannula 24 is coupled to the supplemental outflow port 12. The inflow cannula 20 is dimensioned to extend through the wall of the left atrium such that its distal end is disposed within the left ventricle. The outflow cannula 22 is dimensioned to extend through the wall of the aorta. Under the direction of the pump 10, blood may thus be withdrawn from the left ventricle and re-directed into the aorta, effectively bypassing the aortic valve, as may be required for various cardiac surgery procedures. In accordance with one embodiment of the present invention, the perfusion catheter 24 is dimensioned to extend into a blood vessel 30 on the exterior of the heart. More specifically, with combined reference to FIGS. 2 and 3, the perfusion catheter 24 is preferably to be positioned within the blood vessel 30 such that the distal end 26 extends past a damaged or diseased section 32 of the blood vessel 30, which is to be bypassed (such as via a coronary artery bypass graph (CABG) procedure) removed, or otherwise treated. In practice, the target vessel 30 will be occluded upstream of the damaged or diseased section 32, the occlusion being shown generically at 40.

According to the present invention, positioning the distal end 26 of the perfusion catheter 24 as shown provides the ability to deliver oxygenated blood within the vessel 30 to perfuse the heart tissue located downstream from the occlusion 40, such as while the surgeon is performing an anastomosis to bypass the damaged or diseased section 32 in CABG procedures. In one embodiment, the distal end 26 of the perfusion catheter 24 may be equipped with a selectively inflatable balloon or similar occluding structure 28 designed to prevent the flow of blood upstream towards the damaged or diseased section 32. In this fashion, the balloon or occluding structure 28 helps to establish and maintain a bloodless field along a portion of the target blood vessel 30, thereby easing the challenge for the surgeon in performing the anastomosis.

Although shown as part of a left-heart bypass arrangement in FIG. 2, it is to be readily understood that the pump 10 having the supplemental port 12 of the present invention may be used in any number of cannulation arrangements for cardiac surgery. These may include (but are not necessarily limited to) pump and cannula arrangements for providing left-heart and/or right-heart support, such as set forth in U.S. patent application Ser. No. 08/891,456, now U.S. Pat. No. 6,123,725 (assigned to the assignee of the present application and filed on Jul. 11, 1997), the entire contents of which are hereby expressly incorporated herein by reference. When employed as part of a right-heart cannulation system, the pump 10 of the present invention would provide venous blood (withdrawn from the right side of the heart) through the supplemental port 12. Although this venous blood is (by definition) oxygen depleted, this blood supply may nonetheless be helpful in perfusing locations downstream from a surgical site, as even oxygen-depleted blood is better than no downstream blood flow at all. Moreover, while blood pump 10 is shown as a generic centrifugal blood pump, it is to be readily understood that blood pump 10 may comprise any number of blood pumps, including but not limited to the miniature centrifugal blood pump shown and described in U.S. Provisional Patent Application No. 60/178,479 (filed by the assignee of this application on Jan. 26, 2000), the entire disclosure of which is hereby expressly incorporated herein by reference. It should also be appreciated that, although shown and described above in use with the perfusion catheter 24 for tissue perfusion, the supplemental outflow port 12 may have a variety of other uses. These may include (but are not necessarily limited to) use as a pressure tap to determine the pressure of the outflow from the pump 10, as well as for obtaining blood samples, such as for determining blood. gas content.

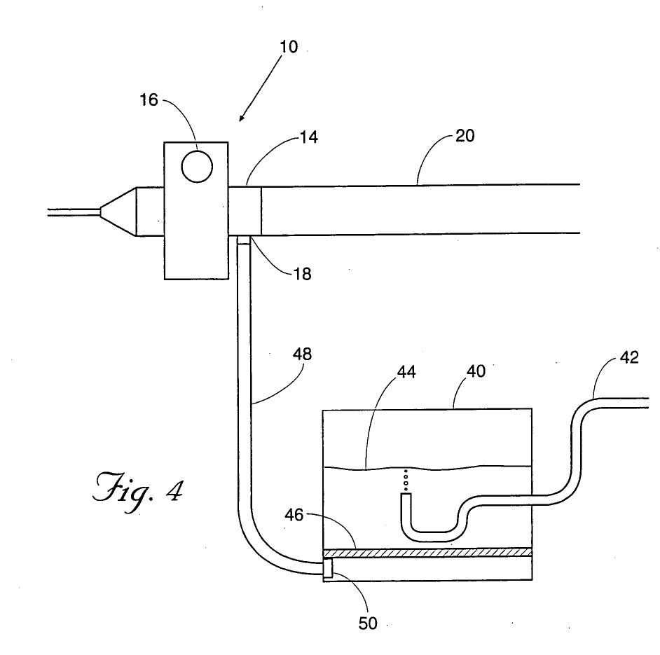

Referring finally to FIG. 4, shown is an alternate embodiment of the present invention. The blood pump 10 is provided with a supplemental inflow port 18 formed as part of the structure defining the main inflow port 14. Under the direction of the motor (not shown), the internally disposed impeller (not shown) will draw blood through the inflow cannula 20, through the pump 10, for delivery out a cannula (not shown) coupled to the main outflow port 16. In accordance with this aspect of the present invention, the supplemental inflow port 18 will provide the ability to draw another fluid into the pump 10 for delivery out the main outflow port 16. For example, during most surgical procedures, blood is drained from the patient's chest cavity through the use of a suction device. Generally, this blood is deposited in a reservoir, such as at 40, via any suitable tubing or fluid conduit 42. The reservoir 40 may serve many purposes, such as for removing any bubbles that develop in the blood during suction and/or filtering the blood 44 in order to recondition it for introduction back into the patient's blood supply. This filtering can be accomplished via any suitable mechanism, such as via the filter shown generally at 46 near the bottom of the reservoir 40. As blood enters the reservoir 40, air will migrate towards the surface of the blood 44 and escape into the atmosphere. A return conduit 48 extends between the reservoir 40 and the supplemental inflow port 18 to allow the reconditioned blood 44 to be withdrawn into the blood supply being delivered into the pump 10. The reservoir 40 may be equipped with a flow regulating mechanism (such as check-valve 50) to ensure that the return conduit 48 is occluded in the event the blood 44 within the reservoir 40 drops below a predetermined level.

The pump 10, equipped with the supplemental inflow port 18 according to the present invention, also advantageously allows the physician to infuse any of a variety of fluids into the blood stream of the patient. As well as the infusion of reconditioned or recaptured blood as shown in FIG. 4, it may be necessary to infuse fluids or substances such as saline and/or various drugs into the patient. The supplemental inflow port 18 of the present invention also provides the ability to deliver these fluids in large quantities and in quick fashion, which may be required in emergency situations where such actions must be taken to save the life of the patient.

As will be appreciated, other combination of the various methods and elements can be used as appropriate. For example, the blood pump of the present invention may be coupled to an oxygenator. While the present invention has been described with reference to the aforementioned examples, this description is not intended to be construed in a limiting sense. It should be readily understood that the components disclosed herein should all be made of materials suitable for medical use, which materials are well known in the art. It should also be understood that all aspects of the present invention are not limited to the specific depictions, and that relative proportions and sizing of the components may vary depending upon the particular situation or application.

Various modifications in form and detail of the embodiments shown herein will be apparent to skilled artisans upon reference to this disclosure. It is therefore contemplated that all attendant claims shall cover any such modifications or variations of the described embodiments as following within the true spirit and scope of the present invention.

1. A method of providing cardiovascular support comprising the steps of:
(a) operably connecting a blood pump to a patient;
b) pumping blood from one part of the heart to another part of the heart; and
c) supplying blood through a supplemental port on the blood pump to an artery on the heart during a bypass operation to that artery.

2. A method as in claim 1
wherein the blood passes through an oxygenator.

3. A method as in claim 1
wherein the blood is supplied from the supplemental port to the artery through a catheter.

4. A method as in claim 1 wherein the blood is supplied from the supplemental port to the artery through a cannula.

5. A method of providing cardiovascular support comprising the steps of:
(a) providing a blood pump, the pump having a main inflow port, a main outflow port, and a supplemental outflow port,
(b) coupling an inflow cannula to the main inflow port, coupling an outflow cannula to the main outflow port, and coupling a supplemental outflow cannula to the supplemental outflow port,
(c) introducing a distal end of the inflow cannula into a heart chamber,
(d) introducing a distal end of the outflow cannula into a first blood vessel,
(e) introducing a distal end of the supplemental outflow cannula into a second blood vessel different from the first blood vessel, and
(f) operating the pump to intake blood from the heart chamber through the inflow cannula and to output blood to the first blood vessel through the outflow cannula and to the second blood vessel through the supplemental outflow cannula.

6. A method as in claim 5
wherein the distal end of the inflow cannula is introduced into the left ventricle.

7. A method as in claim 5
wherein the distal end of the outflow cannula is introduced into the aorta.

8. A method as in claim 5
wherein the distal end of the supplemental outflow cannula is introduced into an exterior coronary vessel downstream of a damaged or diseased section of the exterior coronary vessel which is to be bypassed.

9. A method as in claim 8
wherein the supplemental outflow cannula includes an occluding structure to prevent the flow of blood upstream toward the damaged or diseased section.

10. A method as in claim 9
wherein the occluding device is a balloon.

11. A method as in claim 8, further comprising
occluding the exterior coronary vessel upstream of the damaged or diseased section.

12. A method as in claim 5
wherein the pump further includes a supplemental inflow port.

13. A method as in claim 12, further comprising
coupling a supplemental inflow cannula to the supplemental inflow port,
coupling the supplemental inflow cannula to a fluid reservoir, and
intaking fluid from the fluid reservoir through the supplemental inflow cannula.

14. A method as in claim 13
wherein the fluid includes reconditioned or recaptured blood.
